(19) United States
(12) Patent Application Publication
(10) Pub. No.: US 2011/0319929 A1
(43) Pub. Date: Dec. 29, 2011

(54) MEDICAL PRESSURE APPLICATOR DEVICE

(76) Inventor: Dolores H. Grah, Greenfield, IN (US)

(21) Appl. No.: 13/227,706

(22) Filed: Sep. 8, 2011

Related U.S. Application Data
(63) Continuation-in-part of application No. 13/006,513, filed on Jan. 14, 2011, now Pat. No. 8,033,488.
Application No. 13/006,513 is a continuation of application No. 12/144,138, filed on Jun. 23, 2008, now Pat. No. 7,896,273.
(60) Provisional application No. 60/958,694, filed on Jul. 9, 2007.

(51) Int. Cl. A61B 17/00

(52) U.S. Cl. 606/201

(57) ABSTRACT

A medical pressure applicator device includes an elongate handle and a shaft. The handle has a first end, a second end, and a first axis. The shaft has an offset portion with a second axis and a work portion with a third axis. The offset portion defines a proximal end of the shaft that is connected to the handle at the second end. The work portion defines a distal end of the shaft with the third axis being substantially perpendicular to the first axis. A distance between the first axis and the distal end along the third axis is in a range of about 8.5 to about 15.5 centimeters. The device is configured to transfer a working force from the elongate handle to the distal end of the work portion of the shaft. In some embodiments, a disposable tip body is received over the distal end of the shaft.

### CROSS REFERENCE TO RELATED APPLICATIONS

This is a continuation-in-part of U.S. patent application Ser. No. 13/006,513 filed Jan. 14, 2011, which is a continuation of U.S. patent application Ser. No. 12/144,138 filed Jun. 23, 2008, now U.S. Pat. No. 7,896,273, which is a non-provisional application based upon U.S. Provisional Patent Application Ser. No. 60/958,694, that is entitled—“Pill pro” pill crusher—, filed Jul. 9, 2007, which are incorporated herein by reference.

### MICROFICHE APPENDIX

None.

### GOVERNMENT RIGHTS IN PATENT

None.

### BACKGROUND OF THE INVENTION

1. Field of the Invention

The present invention relates to medical devices, and, more particularly, to a medical pressure applicator device.

2. Description of the Related Art

Part of the Healthy People 2010 campaign is improved medication safety. Currently many hospitals barcode scan the patient and the medication to correlate the medication with the patient. At that point of care, it is desirable that the medication not be taken out of the patient's room, so as to reduce the chance of error.

One type of medication is a pill, also sometimes referred to as a tablet, which has a solid form. Some people, such as those with swallowing difficulties or a strong gag reflex, find it difficult to swallow a pill. It is known in the art to use pill crushers for crushing the pills, so as to convert the pill medications into powder form, which may be more easily ingested.

A typical pill crusher includes multiple parts, movable or a combination of movable and stationary parts, which interact to crush the pill. One such pill crusher, as disclosed in U.S. Pat. No. 6,637,685, has an arm having a pressure exerting head extending over and along an elongate base having a spring loaded rotational anvil, with a pivotal connection between the base and one end of the arm. In use, a rotational movement of the anvil member causes a pill to be both crushed and also simultaneously ground between the pressure and anvil members, which have serrated opposed faces.

It has been found, however, that in a hospital setting, some such pill crushers may be awkward to use. Also, some such pill crushers may require a sizeable area on which to operate, which can be a disadvantage in cramped quarters, such as in a hospital room. Also, some such pill crushers are not readily portable due to their overall size and/or weight. As used herein, the term “portable” means an object that has a size and weight that permits the object to be easily and completely inserted into a pocket of a typical medical garment, and without causing any discomfort to the person wearing the garment, such as due to weight, protruding edges, outside dimensions, etc.

Also, some pill crushers used by nurses in hospitals or nursing homes are kept in the medication room because they are too expensive for there to be one in each patient's room. Hence, nurses may crush the pills far away from the patient's bedside, leaving room for error.

To address the above, U.S. Pat. No. 7,896,273 discloses an ergonomic portable pill crusher tool that is configured to be completely self-contained, and includes an elongate handle coupled to a work portion having a tip configured to facilitate application of a working force to crush a pill.

Currently in the medical arts there are situations that necessitate application of a force during medical treatment. Such applications include, for example and not by way of limitation, (a) applying pressure to stem blood flow from an artery, e.g., a femoral artery; (b) applying pressure and massage to the lower back of a woman in labor; and (c) applying pressure to an area of the abdomen to assist in intestinal maneuvering, e.g., during an endoscopic procedure. In each of the applications identified above, the typical method of applying a force involves the use of a gloved hand in applying a direct pressure to the relevant portion of the patient's body. However, such direct application of force using the practitioner's hand can be very tiring and painful and potentially creating long-term damage to the hand and wrist of the practitioner if the pressure application lasts for an extended period of time.

What is needed in the art is a medical pressure applicator device configured for use in applying a localized pressure to a patient, which is comfortable to both the user and the patient, and which is simple to use, easily transportable, and cost effective.

### SUMMARY OF THE INVENTION

The present invention provides a medical pressure applicator device configured for use in applying a localized pressure to a patient, which is comfortable to both the user and the patient, and which is versatile, simple to use, easily transportable, and cost effective. Also, the patient pressure applicator device of the present invention is not limited to a fixed position, and is maneuverable to adjust as needed.

The invention, in one form thereof, is directed to a medical pressure applicator device configured for use in applying localized pressure. The device includes an elongate handle and a shaft. The handle has a first end, a second end, and a first axis. The second end is spaced apart from the first end along the first axis. The shaft has an offset portion with a second axis and a work portion with a third axis. The offset portion defines a proximal end of the shaft that is connected to the elongate handle at the second end. The work portion defines a distal end of the shaft with the third axis being substantially perpendicular to the first axis, wherein a distance between the first axis and the distal end along the third axis is in a range of about 8.5 to about 15.5 centimeters. The medical pressure applicator device is configured to transfer a working force from the elongate handle to the distal end of the work portion of the shaft to apply a localized pressure when the working force is applied to the elongate handle by a user.

The invention, in another form thereof, is directed to a medical pressure applicator device for use in applying localized pressure to portion of a body of a patient. The device includes an applicator bar and a disposable tip body. The applicator bar has an elongate handle with a handle axis, and a shaft having a proximal end and a distal end. The proximal end is coupled to the elongate handle. The shaft has a work portion with a work axis arranged substantially perpendicular to the handle axis. The work portion terminates at the distal end of the shaft. The disposable tip body has a base and a side wall extending from the base. The side wall and the base are configured to define a closed-end aperture. The closed-end aperture is configured to receive the distal end in a releasable attachment to releasably mount the disposable tip body to the work portion of the shaft. The base has an exterior portion that defines a pressure application area configured to apply the localized pressure to a region of the patient.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of an ergonomic portable pill crusher system configured in accordance with an embodiment of the present invention, with a portion of the cups broken away to expose the respective bottom surface and side wall.

FIG. 2 is a side view of the ergonomic portable pill crusher tool of the ergonomic portable pill crusher system of FIG. 1, with the optional handle sleeve removed.

FIG. 2A is a sectional view of the ergonomic portable pill crusher tool of the ergonomic portable pill crusher system of FIG. 1 taken along line 2A-2A.

FIG. 2B is a sectional view of the work portion of the pill crusher tool of FIG. 1 taken along line 2B-2B.

FIG. 3A is a side view of another embodiment of the tool of FIG. 1, having a work portion adapted for use in general medical pressure application, and having a cylindrical side surface that transitions to a planar pressure application surface via a radius.

FIG. 3B is a side view of another embodiment of the tool of FIG. 1, having a work portion adapted for use in general medical pressure application, and having a cylindrical side surface that transitions to a planar pressure application surface via a radius.

FIG. 3C is a side view of another embodiment of the tool of FIG. 1, having a work portion adapted for use in general medical pressure application, and having a flared side surface that transitions to a planar pressure application surface via a radius.

FIG. 3D is a side view of another embodiment of the tool of FIG. 1, having a work portion adapted for use in general medical pressure application, and having a flared side surface that transitions to a concave pressure application surface via a radius.

FIG. 3E is a side view of another embodiment of the tool of FIG. 1, having a work portion adapted for use in general medical pressure application, and having a flared side surface that transitions to a convex pressure application surface via a radius.

FIG. 3F is an end view depicting an area shape for the tool of FIGS. 3A-3E, having a circular or oval pressure application surface area.

FIG. 3G is an end view depicting an area shape for the tool of FIGS. 3A-3E, having a substantially polygonal surface area.

FIG. 3H is an end view of a special case of the polygonal surface area of FIG. 3G, depicting an area shape for the tool of FIGS. 3A-3E, having a substantially rectangular surface area.

FIG. 31 shows a side view of a portion of the medical pressure applicator device of FIG. 3C, having a cushioning layer.

FIG. 4 is a side view of an embodiment of a medical pressure applicator device that includes an applicator bar and a disposable tip body.

FIG. 4A shows a side view of a portion of the medical pressure applicator device of FIG. 4, having a cushioning layer.

FIG. 5A is a side view of an embodiment of the disposable tip body of FIG. 4 having a flared side surface that transitions to a planar surface at the distal end via a radius.

FIG. 5B is a section view of the disposable tip body of FIG. 5A, taken along line 5B-5B of FIG. 5A.

FIG. 6A is a side view of an embodiment of the disposable tip body of FIG. 4 having a flared side surface that transitions to a concave surface at the distal end via a radius.

FIG. 6B is a section view of the disposable tip body of FIG. 6A, taken along line 6B-6B of FIG. 6A.

FIG. 7A is a side view of an embodiment of the disposable tip body of FIG. 4 having a flared side surface that transitions to a convex surface at the distal end via a radius.

FIG. 7B is a section view of the disposable tip body of FIG. 7A, taken along line 7B-7B of FIG. 7A.

FIG. 8A is a side view of an embodiment of the disposable tip body of FIG. 4 having a flared side surface that transitions to a planar surface having bump protrusions at the distal end.

FIG. 8B is a section view of the disposable tip body of FIG. 8A, taken along line 8B-8B of FIG. 8A.

FIG. 9A is a side view of an embodiment of a disposable tip body having a cylindrical side surface that transitions to a planar surface at the distal end via a radius.

FIG. 9B is a section view of the disposable tip body of FIG. 9A, taken along line 9B-9B of FIG. 9A.

FIG. 10A is a perspective view depicting a shape for the disposable tip body of FIGS. 5A-8A, having a circular or oval pressure application surface area.

FIG. 10B is an end view of the disposable tip body of FIG. 10A.

FIG. 11A is a perspective view depicting a shape for the disposable tip body of FIGS. 5A-8A, having a substantially polygonal pressure application surface area.

FIG. 11B is an end view of the disposable tip body of FIG. 11A.

FIG. 12A is a perspective view of a special case of the substantially polygonal disposable tip body of FIG. 11A, having a substantially rectangular pressure application surface area.

FIG. 12B is an end view of the disposable tip body of FIG. 12A.

FIG. 13 is an end view of the disposable tip body of FIG. 8A having bump protrusions arranged on the circular or oval pressure application surface area of FIG. 10B.

Corresponding reference characters indicate corresponding parts throughout the several views. The exemplifications set out herein illustrate embodiments of the invention, and such exemplifications are not to be construed as limiting the scope of the invention in any manner

### DETAILED DESCRIPTION OF THE INVENTION

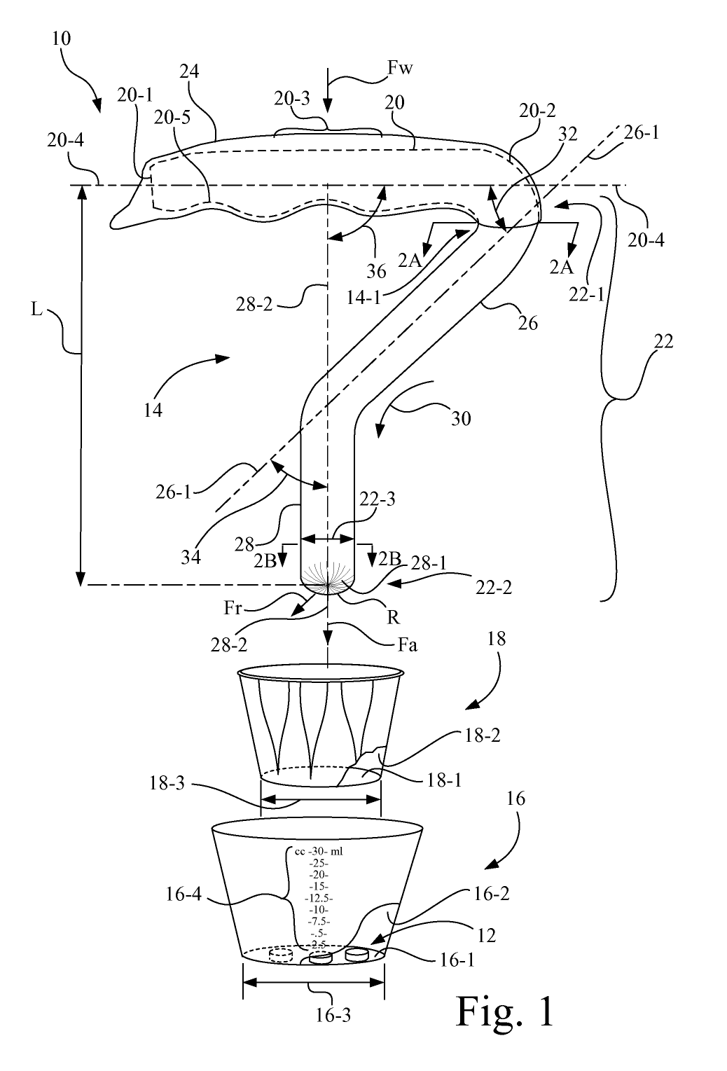

Referring now to the drawings, and more particularly to FIG. 1, there is shown an ergonomic portable pill crusher system 10 configured in accordance with an embodiment of the present invention for crushing one or pills, such as the plurality of pills 12. The ergonomic portable pill crusher system 10 includes an ergonomic portable pill crusher tool 14, a first cup 16 for holding the plurality of pills 12 during a pill crushing operation, and a second cup 18 for covering over the plurality of pills 12 held by first cup 16 during the pill crushing operation.

First cup 16, for example, may be formed from a transparent plastic, and includes a bottom surface 16-1 and a side wall 16-2 extending upwardly from bottom surface 16-1. Bottom surface 16-1 of first cup 16 has a diameter 16-3. Side wall 16-2 may include measurement indicia 16-4 for measuring a volume of crushed medication in powder form resulting from the crushing operation, i.e., the crushing of the plurality of pills 12.

Second cup 18, for example, may be a standard three-fourths ounce paper souffle cup. Second cup 18 includes a bottom surface 18-1 and a side wall 18-2 extending upwardly from bottom surface 18-1. Bottom surface 18-1 of second cup 18 has a diameter 18-3 sized to be slightly smaller that diameter 16-3 of first cup 16.

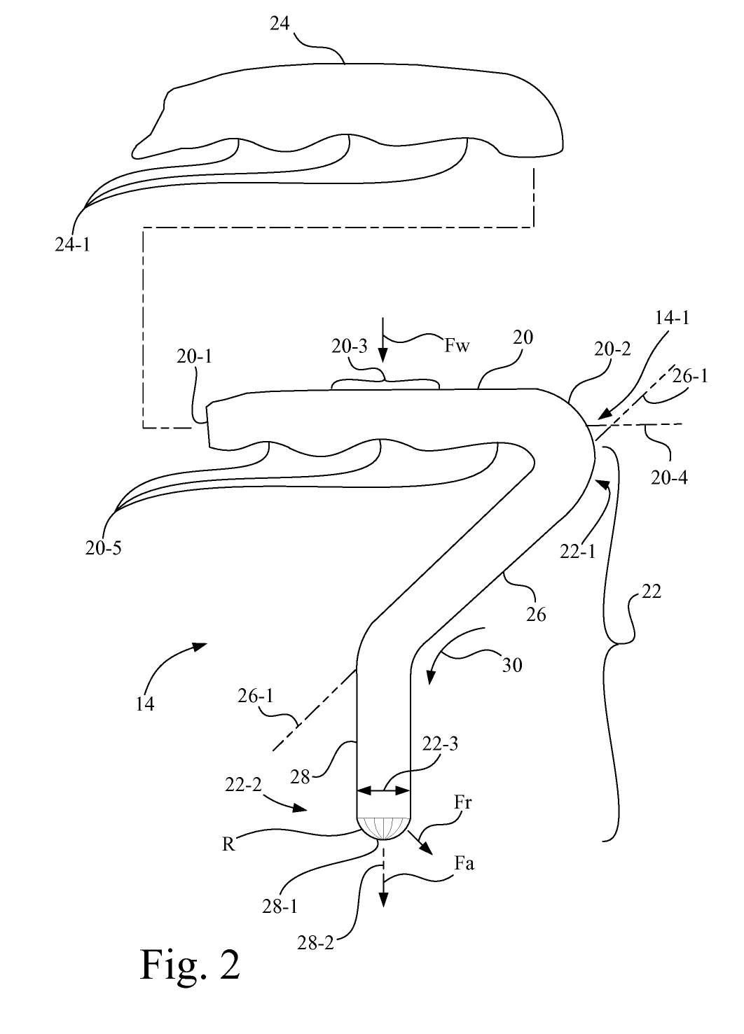

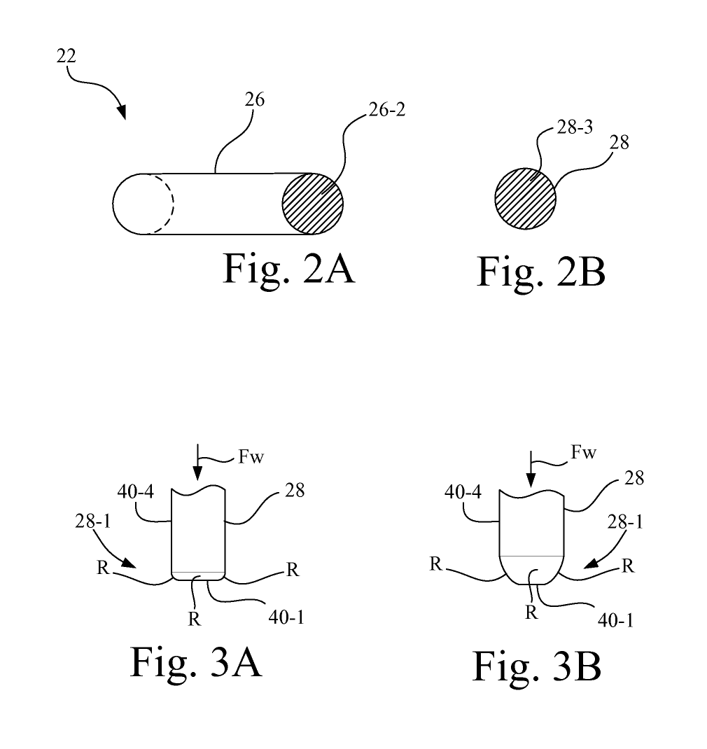

Referring also to FIGS. 2, 2A and 2B, ergonomic portable pill crusher tool 14 includes an elongate handle 20, a curved shaft 22, and optionally, a sleeve 24 fitted over elongate handle 20. Sleeve 24, for example, may be formed from an elastic material, such as rubber. Sleeve 24 may optionally include a plurality of handhold recesses 24-1 to accommodate grasping of elongate handle 20 by the fingers of a user.

Elongate handle 20 has a first end 20-1, a second end 20-2, a central portion 20-3, and a first axis 20-4. First end 20-1 is spaced apart from second end 20-2 along first axis 20-4, with central portion 20-3 being located midway between first end 20-1 and second end 20-2. As used herein, the term “central portion” means the central third of the entire lateral extent of elongate handle 20. Elongate handle 20 optionally may include a plurality of handhold recesses 20-5 to accommodate grasping of elongate handle 20 by the fingers of a user.

Shaft 22 has an offset portion 26 and a work portion 28. Offset portion 26 defines a proximal end 22-1 of shaft 22 and work portion 28 defines a distal end 22-2 of shaft 22. Distal end 22-2 of work portion 28 of shaft 22 has a diameter 22-3. Diameter 22-3 may be, for example, in a range of 10 to 20 millimeters, and in one embodiment is 15.9 millimeters (0.625 inches). Distal end 22-2 of work portion 28 of shaft 22 has a tip end 28-1, which in the present embodiment has a convex shape. Tip end 28-1 may be in the form of a smooth outwardly extending curved surface. Alternatively, tip end 28-1 may be blunt, i.e., flat, if desired.

Offset portion 26 is connected at proximal end 22-1 of shaft 22 to second end 20-2 of elongate handle 20. Offset portion 26 of shaft 22 may be integrally formed with elongate handle 20 such that shaft 22 and elongate handle 20 are formed as an integral one-piece device. Alternatively, it is contemplated that proximal end 22-1 of shaft 22 and second end 20-2 of elongate handle 20 may be threaded and screwed together to facilitate a threaded attachment thereof.

In the present embodiment, offset portion 26 transitions to work portion 28 in a direction 30 from proximal end 22-1 to distal end 22-2 of shaft 22. Offset portion 26 has a second axis 26-1 that is arranged to be oblique with respect to first axis 20-4 of elongate handle 20 at an angle 32 (less than 90 degrees, and preferably in a range of 30 to 60 degrees) facing central portion 20-3.

Work portion 28 has a third axis 28-2 that is arranged to be oblique with respect to second axis 26-1 of offset portion 26 at an angle 34 (less than 90 degrees, and preferably in a range of 30 to 60 degrees) facing away from central portion 20-3, such that an extent of work portion 28 along third axis 28-2 is substantially perpendicular at angle 36 to first axis 20-4 of elongate handle 20 at central portion 20-3 of elongate handle 20. As used herein, the term substantially perpendicular means an angle of 90 degrees, plus or minus 2 degrees. A length L of the portable pill crusher tool 14 from first axis 20-4 to distal end 22-2 of work portion 28 of shaft 22 along axis third axis 28-2 may be in a range of about 8.5 to about 15.5 centimeters (about 3.5 inches to about 6 inches). As used herein, the term “about” in relation to a distance is a tolerance range of plus or minus 15 percent.

Tip end 28-1 of distal end 22-2 of work portion 28, such as when shaped as a convex tip, facilitates application of an axial force Fa and radial forces Fr, with respect to third axis 28-2, to the one or more pills, e.g., the plurality of pills 12 located in first cup 16. As shown in FIG. 1, second cup 18 is inserted into first cup 16, and interposed between tip end 28-1 of distal end 22-2 of shaft 22 and the respective pill(s) located in first cup 16.

In some embodiments, ergonomic portable pill crusher tool 14 may include a flexure portion 14-1 located between elongate handle 20 and work portion 28 of shaft 22. Flexure portion 14-1 is configured to facilitate a radial flexure of work portion 28 along third axis 28-2 when a working force Fw is applied by a user along third axis 28-2 via elongate handle 20. Working force Fw may be primarily an axial force, but may include radial force components due to a rocking motion of ergonomic portable pill crusher tool 14 during use. Flexure portion 14-1 may be formed as a cantilever union of elongate handle 20 and shaft 22.

An amount of flexure resulting from flexure portion 14-1 may be defined based on the material forming offset portion 26 of shaft 22 and a cross-sectional area 26-2 of offset portion 26, and thus form flexure portion 14-1 in offset portion 26. The material forming shaft 22, including offset portion 26, may be for example a plastic or a metal (e.g., stainless steel). In one embodiment, for example, the amount of flexure at angle 32 may be selected to be in a range of two to five degrees upon application of 10 pounds of axial working force Fw to elongate handle 20. In another embodiment, for example, the amount of flexure at angle 32 may be selected to be in a range of zero to five degrees upon application of 10 pounds of axial working force Fw to elongate handle 20.

In embodiments wherein shaft 22 is made of plastic, the type of plastic may be selected to withstand the applied force Fw while providing the desired flexure (if any) based on the desired cross-sectional area 26-2. Alternatively, the cross-sectional area 26-2 may be selected to withstand the applied force Fw while providing the desired flexure (if any) based on the desired type of plastic. Also, the type of plastic is selected so as to not deteriorate when cleaned with normal medical disinfectant cleaning solutions, e.g., alcohol or bleach based solutions.

Likewise, in embodiments wherein shaft 22 is made of metal, the type of metal may be selected to withstand the applied force Fw while providing the desired flexure (if any) based on the desired cross-sectional area 26-2. Alternatively, the cross-sectional area 26-2 may be selected to withstand the applied force Fw while providing the desired flexure (if any) based on the desired type of metal. Also, the type of metal is selected so as to not deteriorate when cleaned with normal medical disinfectant cleaning solutions, e.g., alcohol or bleach based solutions.

During operation, a user positions one or more pills, e.g., the plurality of pills 12 shown in FIG. 1, into first cup 16, e.g., a standard 30 milliliter plastic cup. Second cup 18, e.g., a standard three-fourths ounce paper souffle cup, is placed in first cup 16 over the pill(s) so as to prevent pill fragments from flying out of first cup 16, and to prevent pill fragments from adhering to tip end 28-1 of shaft 22 of ergonomic portable pill crusher tool 14.

The user inserts distal end 22-2 of ergonomic portable pill crusher tool 14 into second cup 18 with tip end 28-1 contacting bottom surface 18-1 of second cup 18. The user then applies a firm working force Fw to elongate handle 20 of ergonomic portable pill crusher tool 14. Working force Fw is preferably a combination of a linear (axial) pressing force and a rocking/rotating action applied to elongate handle 20. Working force Fw results in axial force Fa and radial forces Fr, with respect to third axis 28-2, applied by tip end 28-1 to crush the pill(s), e.g., the plurality of pills 12, positioned between first cup 16 and second cup 18. Diameter 22-3 of distal end 22-2 of work portion 28 of shaft 22 is preferably less than half of diameter 18-3 of bottom surface 18-1 of second cup 18 so as to permit tip end 28-1 of distal end 22-2 of shaft 22 freedom to move side-to-side in second cup 18 along bottom surface 18-1 within side wall 18-2 of second cup 18. The pill particles may be gently agitated during the crushing operation by moving second cup 18, e.g., side-to-side or rotationally, relative to first cup 16.

As second cup 18 is removed from first cup 16, any particles of the crushed medication adhering to second cup 18 may be brushed off into first cup 16. Also, any particles of the crushed medication adhering to first cup 16 may be dislodged from bottom surface 16-1 and/or side wall 16-2 by tapping bottom surface 16-1 and/or side wall 16-2, e.g., on a table top or with an object, such as ergonomic portable pill crusher tool 14.

In the embodiments described above, for example, the dimensional proportions of ergonomic portable pill crusher tool 14 shown in FIGS. 1-2B are exemplary of one embodiment, and those skilled in the art will recognize that variations from the dimensional proportions shown may be made without departing from the scope of the present invention. For example, one skilled in the art will recognize that the size of elongate handle 20 may be increased or decreased from that shown to accommodate the handle size desired by respective users. For example, the handle size may come in a plurality of sizes, such as small, medium and large, to more precisely fit the size of the hand of a particular user.

It has been discovered that tool 14 may be used as is or modified, as described below, to perform one or more additional tasks in the medical field, such as that of a medical pressure applicator device 14 that may be used by a medical practitioner, such as a nurse, doctor, physical therapist, etc., to apply localized pressure to a region of a patient's body. Such applications include, for example and not by way of limitation, (a) applying pressure to stem blood flow from an artery, e.g., a femoral artery; (b) applying pressure and massage to the lower back of a woman in labor; and (c) applying pressure to an area of the abdomen to assist in intestinal maneuvering, e.g., during an endoscopic procedure. Medical pressure applicator device 14 is configured as a self-contained device configured to transfer the working force Fw from the elongate handle 20 to the distal end 22-2 of work portion 28 of shaft 22 to apply a localized pressure when the working force Fw is applied to the elongate handle 20 by a user, e.g., medical practitioner. In the present embodiment, medical pressure applicator device 14 has length L from first axis 20-4 to distal end 22-2 of work portion 28 of shaft 22 along axis third axis 28-2 in a range of about 8.5 to about 15.5 centimeters (about 3.5 inches to about 6 inches).

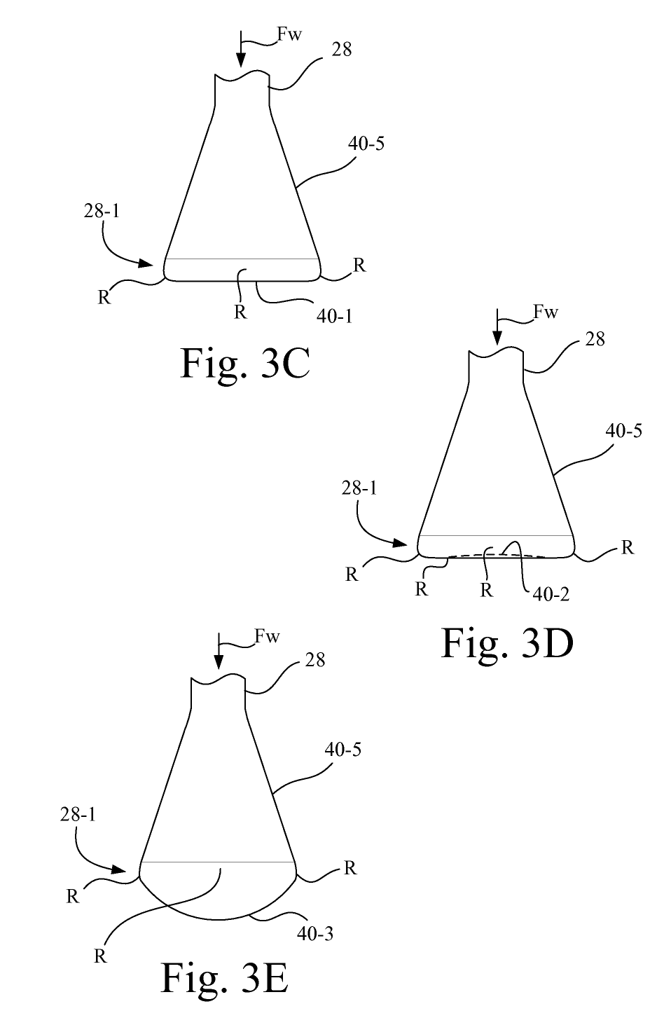

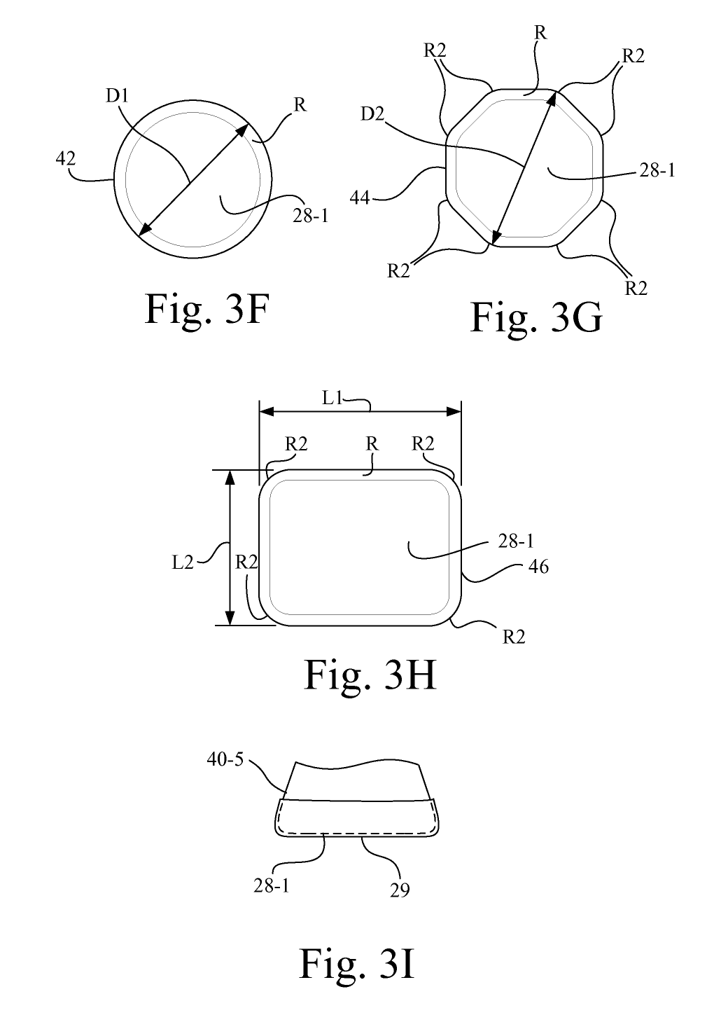

Depending upon the application, the medical practitioner may select a medical pressure applicator device 14 having a tip shape better suited to performing the task at hand. For example, it is contemplated that the contact area of tip end 28-1 of work portion 28 may have a variety of contours, such as having a planar surface 40-1 (e.g., flat) as shown in FIGS. 3A-3C, a concave surface 40-2 as shown in FIG. 3D, or a convex surface 40-3 as shown in FIG. 3E (see also FIGS. 1 and 2). Each of the embodiments of FIGS. 1-3E, includes a surface edge radius R, which may be of various sizes, that transitions from a side surface of work-portion 28 to tip end 28-1 such that no portion of the surface of the tip end 28-1 will have a sharp edge in contact with the patient.

For example, in the embodiments of FIGS. 3A and 3B the side surface shape of work-portion 28 is shown as being cylindrical to form a cylindrical side surface 40-4. In the embodiments of FIGS. 3A and 3B, cylindrical side surface 40-4 smoothly tapers to tip end 28-1 by radius R. In comparing the embodiment of FIG. 3A with that of FIG. 3B, it may be observed that the larger the radius R, the smaller the cross-directional distance across tip end 28-1. Thus, another characteristic of having the tapering surface edge radius R in the embodiments of FIGS. 3A and 3B is the decrease in the tip-to-patient pressure application surface area over that of a cross-sectional area 28-3 of work portion 28 (see FIG. 2B), thus concentrating the working force Fw over a smaller surface area than that of the cross-sectional area 28-3 of work portion 28.

In the embodiments of FIGS. 3C, 3D and 3E, the shape of the side surface of work portion 28 in a direction from handle 20 toward tip end 28-1 is flared outwardly to form a flared side surface 40-5. The flared side surface 40-5 transitions to a pressure application surface area via a radius R. For many medical pressure application tasks, the flared side surface 40-5 advantageously increases the tip-to-patient pressure application surface area over that of a cross-sectional area 28-3 (see FIG. 2B) of work portion 28, thus distributing the working force Fw over a larger surface area than that of the cross-sectional area 28-3 of work portion 28.

With further reference to FIGS. 3F-3H, tip end 28-1 of work portion 28 in each of FIGS. 3A-3E may be configured such that tip end 28-1 provides a desired predefined tip-to-patient pressure application surface area size and shape. For example, the area shape may be a circular or oval pressure application surface area 42 (FIG. 3F), or a substantially polygonal pressure application surface area 44 (FIG. 3G) such as a substantially rectangular pressure application surface area 46 (FIG. 3H). Here, the term “substantially” is used as a modification of the primary polygonal shape to include corner radii R2 in addition to surface radii R, so as to smooth each corner, to prevent the creation of any sharp edge or pointed corner that might contact the patient and cause patient discomfort.

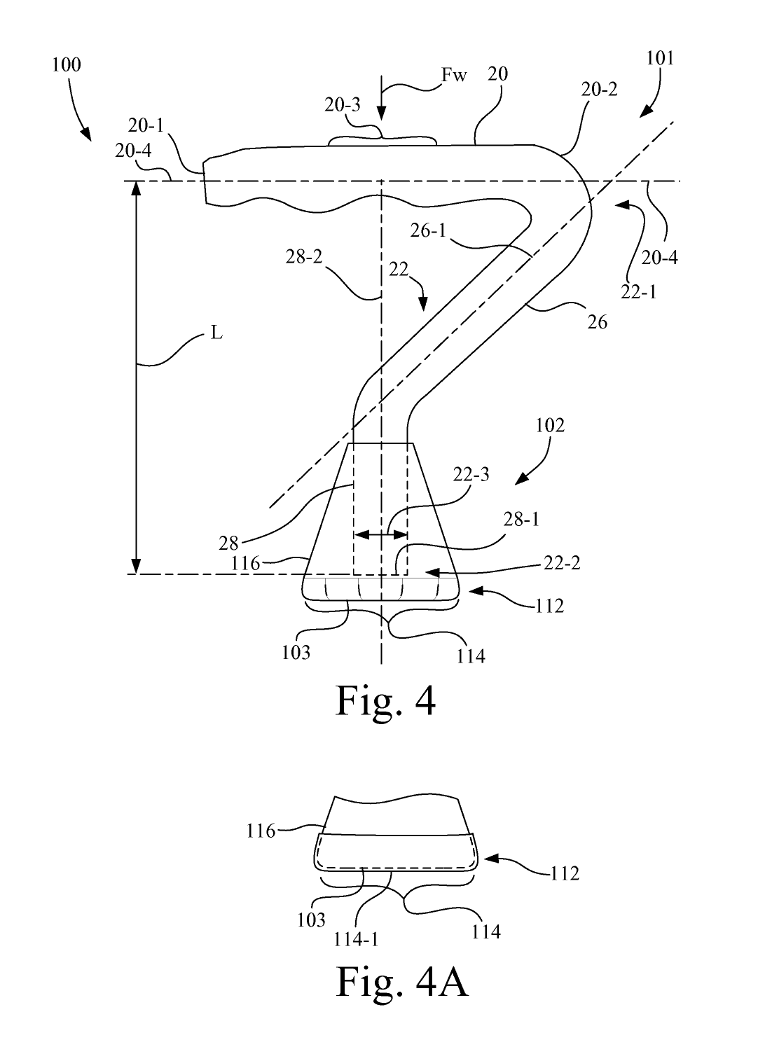

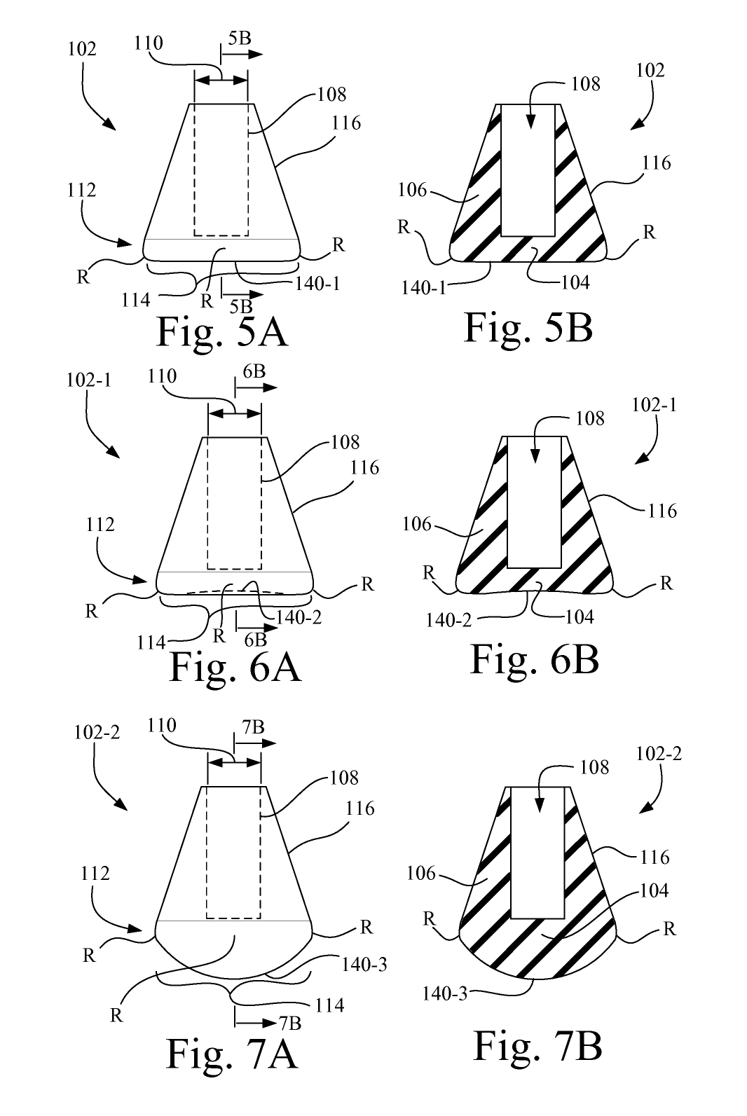

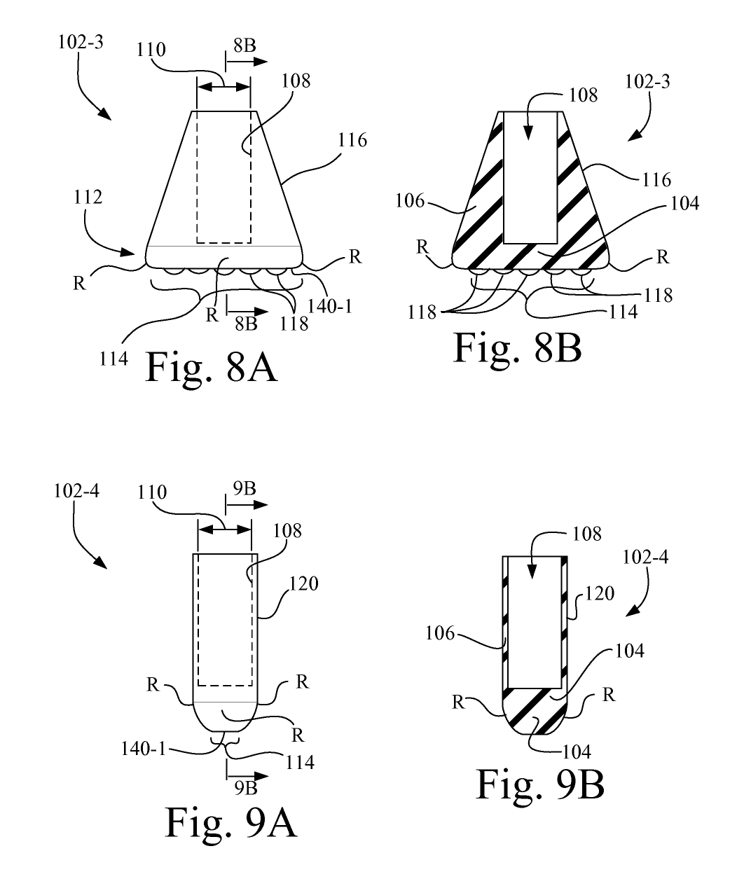

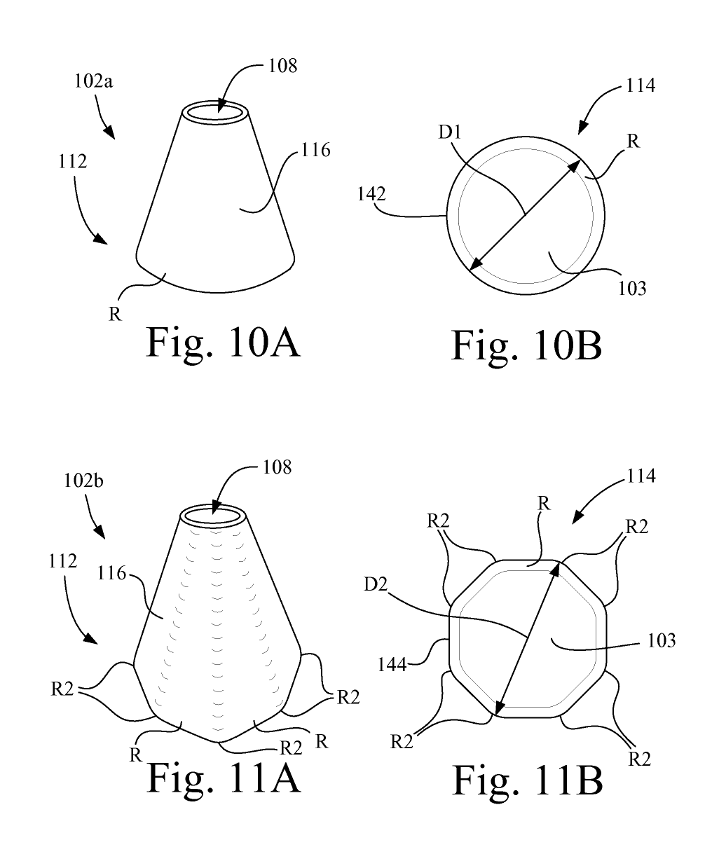

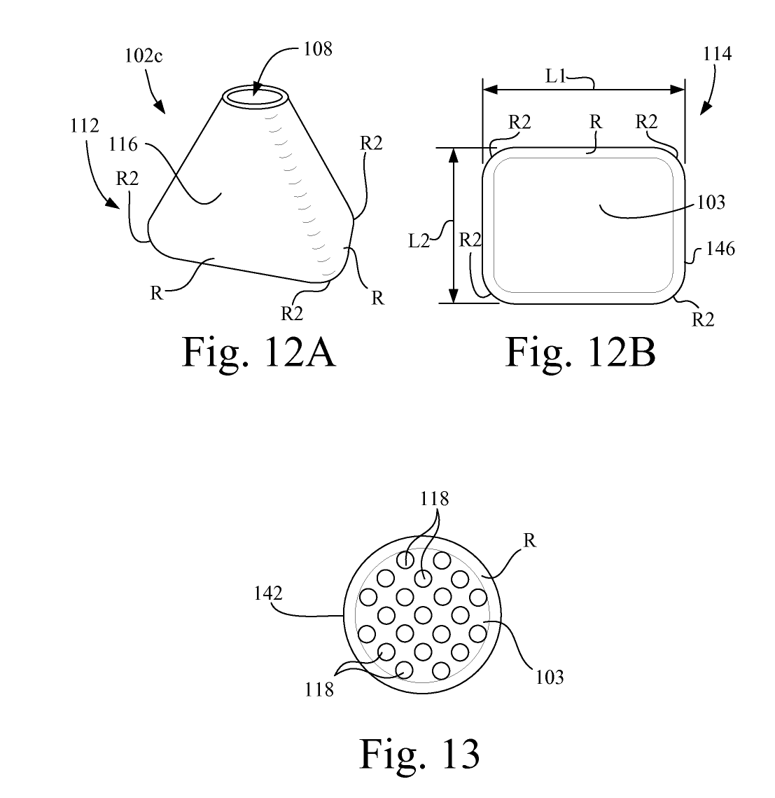

As a further modification of the embodiments of FIGS. 3A-3H, it is contemplated that at least a portion of medical pressure applicator device 14 that comes in contact with the patient may be covered with a cushioning layer 29, e.g. foam rubber, cloth, etc., as illustrated in FIG. 31.

A suitable configuration of tip end 28-1 for applying pressure to stem blood flow from an artery, e.g., a femoral artery, for example, may be obtained by a configuration having the flared side surface 40-5 with the planar tip surface 40-1 as depicted in FIG. 3C, and with a substantially rectangular tip-to-patient pressure application surface area 46 as shown in FIG. 3H. The substantially rectangular pressure application surface area 46 may have, for example, a length L1 (e.g., a longest side length) in a range of abut 5 centimeters (cm) to about 8 cm (about 2 inches to about 3 inches) and a length L2 (e.g., a shorter side length) in a range of about 2.5 cm to about 5.1 cm (about 1 inch to about 2 inches).

A suitable configuration of tip end 28-1 for applying pressure and massage to the lower back of a woman in labor, for example, may be obtained by using a configuration having the flared side surface 40-5 with a planar tip surface 40-1 as depicted in FIG. 3C, or with convex tip surface 40-3 as depicted in FIG. 3E, and with a circular tip-to-patient pressure application surface area 42 as depicted in FIG. 3F. The circular pressure application surface area 42 may have, for example, a longest cross-dimension (e.g., diameter) D1 of about 6.3 cm to about 8.9 cm (about 2.5 inches to about 3.5 inches). Alternatively, the polygonal (e.g., octagonal as shown) tip-to-patient pressure application surface area 44 as depicted in FIG. 3G may be utilized, having for example a longest cross-dimension D2 of about 6.3 cm to about 8.9 cm (about 2.5 inches to about 3.5 inches). Also, it is contemplated that for such massage tasks, a pair of medical pressure applicator devices 14 (one in each hand) may be used simultaneously by the user, e.g., practitioner or labor coach.

A suitable configuration of tip end 28-1 for applying pressure to an area of the abdomen to assist in intestinal maneuvering, e.g., during an endoscopic procedure, for example, may be obtained by using a configuration having the flared side surface 40-5 with planar tip surface 40-1 as depicted in FIG. 3C, or with convex tip surface 40-3 as depicted in FIG. 3E, and with a circular tip-to-patient pressure application surface area 42 as depicted in FIG. 3F. The circular pressure application surface area 42 may have, for example, a longest cross-dimension (e.g., diameter) D1 of 3.8 cm to 6.4 (about 1.5 inches to about 2.5 inches). Alternatively, the polygonal (e.g., octagonal as shown) tip-to-patient pressure application surface area 44 as depicted in FIG. 3G may be utilized, having for example a longest cross-dimension (e.g., diameter) D2 of about 3.8 cm to about 6.4 (about 1.5 inches to about 2.5 inches).

Each tip configuration described above may be incorporated into a respective disposable tip body, as depicted for example in FIGS. 4-13, which is configured to be removably mounted to the work portion 28 of the shaft 22 of a medical pressure applicator device 100. Thus, as an alternative to having a plurality of medical pressure applicator devices 14, each having a different tip configuration as illustrated in FIGS. 3A-3H, it is contemplated that in some instances it may be more convenient for the practitioner to carry a single medical pressure applicator device 100, configured to receive one of a plurality of disposable tip bodies having shapes which may generally correspond to the shapes depicted in FIGS. 3A-3H. The medical pressure applicator device 100 with the plurality of disposable tips may be provided as kit.

Referring now to FIG. 4, medical pressure applicator device 100 may have features in common with tool 14, which for convenience may be referred to by the same name and element number. Thus, in the present embodiment, medical pressure applicator device 100 includes handle 20 and shaft 22 that together form an applicator bar 101, to which a disposable tip body 102 is removably mounted. Medical pressure applicator device 100 is configured to be completely self-contained, and configured to transfer a working force Fw from handle 20 to a pressure application area at the distal end 103 of said disposable tip body 102 to apply a localized pressure to a region of a patient when the working force Fw is applied to the handle 20 by the user.

As shown in FIG. 4A, it is contemplated that at least a portion of the disposable tip body 102 that comes in contact with a patient may be covered with a cushioning layer 114-1, e.g., foam rubber, cloth, etc.

In the present embodiment, handle 20 has first end 20-1, second end 20-2, central portion 20-3, and first axis 20-4, as more fully described above. Also, in the present embodiment, shaft 22 has proximal end 22-1, distal end 22-2, offset portion 26 and work portion 28, as more fully described above. Work portion 28 is mechanically coupled to handle 20, and may be formed integral therewith in forming applicator bar 101. Applicator bar 101 may be constructed, for example, from metal or plastic, as described above with respect to tool 14. A terminal end of work portion 28 defines the distal end 22-2 of shaft 22. In the present embodiment, a distance L between the first axis 20-4 and the distal end 22-2 at tip end 28-1 along the third axis 28-2 is in a range, for example, of about 8.5 to about 15.5 centimeters (about 3.5 inches to about 6 inches).

Work portion 28 has a diameter 22-3, near distal end 22-2. Diameter 22-3 may be, for example, in a range of about 10 to about 20 millimeters (about 0.4 inches to about 0.8 inches), and in one embodiment is 15.9 millimeters (0.625 inches) to define the cross-sectional area 28-3 (see FIG. 2B) of work portion 28 of shaft 22. Distal end 22-2 of work portion 28 of shaft 22 includes tip end 28-1, which in the present embodiment has a blunt planar shape. However, it is contemplated that tip end 28-1 may include a radius, as shown for example in FIG. 3A.

Referring also to FIGS. 5A and 5B, disposable tip body 102 is preferably constructed as a single piece article. Disposable tip body 102 is formed from a firm durable and/or resilient material, such as hard rubber or plastic, and may be formed using a molding process. Disposable tip body 102 may be pre-sterilized and sealed in air-tight packaging prior to use, or may be sterilized prior to use.

Disposable tip body 102 includes a base 104 and a side wall 106 extending from base 104. Side wall 106 and base 104 are configured to define a closed-end aperture 108. The closed-end aperture 108 may be formed in disposable tip body 102, for example, during a molding process, or may be formed as a bore in disposable tip body 102. Closed-end aperture 108 is configured to receive the distal end 22-2 of work portion 28 to provide releasable attachment of disposable tip body 102 to work portion 28 of shaft 22. For example, a diameter 110 of closed-end aperture 108 may be selected to be slightly less than the diameter 22-3 of work portion 28 to as to create a friction fit between disposable tip body 102 and work portion 28 of shaft 22. Alternatively, it is contemplated that the releasable attachment of disposable tip body 102 to work portion 28 of shaft 22 may be achieved by a threaded connection, a temporary adhesive (e.g., thermal release), or mechanical fasteners.

Base 104 has an exterior portion 112 that defines a pressure application area 114, e.g., one of pressure application surface areas 142, 144, 146 illustrated in FIGS. 10B, 11B, and 12B, configured to apply a localized pressure to a region of a patient. The exterior portion 112 at the pressure application area 114, and thus the pressure application area 114 itself, has rounded edges with radii R and rounded corners with radii R2 (see, e.g., FIGS. 10B, 11B, and 12B). Each occurrence of radius R may, but need not be, the same dimension. Also, as between the several embodiments, radii R may, but need not be, the same dimension. Also, radii R2 may, but need not be, the same dimension.

In the present embodiment, disposable tip body 102 has a flared side surface 116 similar to the flared side surface 40-5 described above, with the flare becoming wider in a direction from handle 20 toward distal end 103 at pressure application area 114 (see FIG. 4). Thus, the amount of flaring of disposable tip body 102 may in part determine the amount of the surface area of pressure application area 114.

In the embodiment of FIGS. 5A and 5B, disposable tip body 102 includes a planar surface 140-1 for applying pressure to the patient. Disposable tip body 102 having planar surface 140-1 may be used in pressure applications, for example, involving applying pressure to stem blood flow from an artery, or applying pressure and massage to the lower back of a woman in labor.

FIGS. 6A and 6B show a variation of disposable tip body 102 as disposable tip body 102-1. Disposable tip body 102-1 differs from disposable tip body 102 by the inclusion of a concave surface 140-2 in contrast to the planar surface 140-1 of disposable tip body 102. Thus, in the embodiment of FIGS. 6A and 6B, the pressure application area 114 (e.g., one of pressure application surface areas 142, 144, or 146 illustrated in FIGS. 10B, 11B, and 12B) of the exterior portion 112 of disposable tip body 102-1 is indented along the third axis 28-2 to form the concave surface 140-2. Disposable tip body 102-1 having concave surface 140-2 may be used in pressure applications, for example, involving a portion of a patient's body having a convex surface feature, such as a biceps muscle.

FIGS. 7A and 7B show another variation of disposable tip body 102 as disposable tip body 102-2. Disposable tip body 102-2 differs from disposable tip body 102 by the inclusion of the convex surface 140-3 in contrast to the planar surface 140-1 of disposable tip body 102. Thus, in the embodiment of FIGS. 7A and 7B, the pressure application area 114 (e.g., one of pressure application surface areas 142, 144, or 146 illustrated in FIGS. 10B, 11B, and 12B) of exterior portion 112 of disposable tip body 102-2 is extended along the third axis 28-2 to form a convex surface 140-3. Disposable tip body 102-2 having the convex surface 140-3 may be used in pressure applications, for example, involving applying pressure and massage to the lower back of a woman in labor.

FIGS. 8A and 8B show another variation of disposable tip body 102 as disposable tip body 102-3. Disposable tip body 102-3 differs from disposable tip body 102 by the inclusion of a plurality of bump protrusions 118 on the substantially planar surface 140-1. Disposable tip body 102-3 having bump protrusions 118 may be used in pressure applications, for example, involving applying pressure and massage to the hands or feet of a patient.

FIGS. 9A and 9B show another variation of disposable tip body 102 as disposable tip body 102-4, which noticeably does not include the flared side surface 116, but rather, has a cylindrical side surface 120. A characteristic of the embodiments of FIGS. 9A and 9B is the decrease in the tip-to-patient pressure application surface area over that of a cross-sectional area 28-3 of work portion 28 (see FIG. 2B), thus concentrating the working force Fw over a smaller surface area than that of the cross-sectional area 28-3 of work portion 28.

FIGS. 10A-12B illustrate various shapes associated with pressure application areas 142, 144, and 146 that may be used in conjunction with any of disposable tip bodies 102, 102-1, 102-2, 102-3, and 12-4. For brevity, however, the various pressure application areas 142, 144, and 146, will be described in relation their respective incorporation into disposable tip body 102 having planar surface 40-1, with the variations identified as disposable tip body 102a, disposable tip body 102b, and disposable tip body 102c.

FIGS. 10A and 10B illustrate disposable tip body 102a having a circular or oval pressure application surface area 142 (circular as shown). As such, in the present embodiment disposable tip body 102a has a frustoconical shape. The pressure application surface area 142 may have, for example, a longest cross-dimension (e.g., diameter) D1 of about 6.3 cm to about 8.9 cm (about 2.5 inches to about 3.5 inches).

FIGS. 11A and 11B illustrate disposable tip body 102b having a substantially polygonal (e.g., octagonal as shown) pressure application surface area 144. Pressure application surface area 144 may have, for example, a longest cross-dimension D2 of about 6.3 cm to about 8.9 cm (about 2.5 inches to about 3.5 inches). Here, the term “substantially” is used as a modification of the primary polygonal shape to include surface edge radii R and corner radii R2, so as to smooth each edge and each corner, to prevent the creation of any sharp edge or pointed corner that might contact the patient.

FIGS. 12A and 12B illustrate a special case of the substantially polygonal disposable tip body 102b, in the form of a disposable tip body 102c having a substantially rectangular pressure application surface area 146. The substantially rectangular pressure application surface area 146 may have, for example, a length L1 (e.g., a longest side length) in a range of abut 5 centimeters (cm) to about 8 cm (about 2 inches to about 3 inches) and a length L2 (e.g., a shorter side length) in a range of about 2.5 cm to about 5.1 cm (about 1 inch to about 2 inches). Again, the term “substantially” is used as a modification of the primary rectangular shape to include surface edge radii R and corner radii R2, so as to smooth each edge and each corner, to prevent the creation of any sharp edge or pointed corner that might contact the patient.

FIG. 13 illustrates a bottom view of the embodiment shown in FIGS. 8A and 8B, having bump protrusions 118 symmetrically arranged across the pressure application surface area 142. However, it is contemplated that bump protrusions 118 may be adapted for use with any of pressure application surface areas 142, 144, and 146, if desired.

While this invention has been described with respect to at least one embodiment, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A medical pressure applicator device configured for use in applying localized pressure, comprising:
an elongate handle having a first end, a second end, and a first axis, said second end being spaced apart from said first end along said first axis; and
a shaft having an offset portion with a second axis and a work portion with a third axis, said offset portion defining a proximal end of said shaft that is connected to said elongate handle at said second end, and said work portion defining a distal end of said shaft with said third axis being substantially perpendicular to said first axis, wherein a distance between said first axis and said distal end along said third axis is in a range of about 8.5 to about 15.5 centimeters,
said medical pressure applicator device being configured to transfer a working force from said elongate handle to said distal end of said work portion to apply a localized pressure when said working force is applied to said elongate handle by a user.

2. The medical pressure applicator device of claim 1, comprising:
a pressure application area at said distal end of said work portion;
a flared side surface at said work portion that is flared outwardly in a direction from said handle toward said distal end of said work portion; and
a rounded edge that transitions from said flared side surface to said pressure application area at said distal end of said work portion.

3. The medical pressure applicator device of claim 1, comprising a pressure application area at said distal end of said work portion, wherein said pressure application area is configured to have a circular or oval shape.

4. The medical pressure applicator device of claim 1, comprising a pressure application area at said distal end of said work portion, wherein said pressure application area is configured to have a substantially polygonal shape.

5. The medical pressure applicator device of claim 1, further comprising a disposable tip body having a base and a side wall extending from said base, said side wall and said base being configured to define a closed-end aperture, said closed-end aperture being configured to receive said distal end to releasably attach said disposable tip body to said work portion of said shaft, said base having an exterior portion that defines a pressure application area configured to apply said localized pressure to a region of a patient.

6. The medical pressure applicator device of claim 5, wherein said side wall has a side surface, said exterior portion having a rounded edge that transitions from said side surface to said pressure application area.

7. The medical pressure applicator device of claim 5, wherein said pressure application area is configured to have a circular or oval shape.

8. The medical pressure applicator device of claim 5, wherein said pressure application area is configured to have a generally polygonal shape.

9. The medical pressure applicator device of claim 5, wherein said pressure application area of said disposable tip body is greater than a cross-sectional area of said work portion of said shaft.

10. The medical pressure applicator device of claim 5, wherein said pressure application area of said disposable tip body is less than a cross-sectional area of said work portion of said shaft.

11. The medical pressure applicator device of claim 5, wherein said pressure application area of said disposable tip body is extended along said third axis to form a convex surface.

12. The medical pressure applicator device of claim 5, wherein said pressure application area of said disposable tip body is indented along said third axis to form a concave surface.

13. A medical pressure applicator device for use in applying localized pressure to portion of a body of a patient, comprising:
an applicator bar having an elongate handle with a handle axis, and a shaft having a proximal end and a distal end, said proximal end being coupled to said elongate handle, said shaft having a work portion with a work axis arranged substantially perpendicular to said handle axis, said work portion terminating at said distal end of said shaft; and
a disposable tip body having a base and a side wall extending from said base, said side wall and said base being configured to define a closed-end aperture, said closed-end aperture being configured to receive said distal end in a releasable attachment to releasably mount said disposable tip body to said work portion of said shaft, said base having an exterior portion with a pressure application area configured to apply localized pressure to a region of the patient.

14. The medical pressure applicator device of claim 13, wherein said pressure application area is configured to have a circular shape.

15. The medical pressure applicator device of claim 13, wherein said pressure application area is configured to have a generally polygonal cross-sectional shape with no sharp corners or sharp edges.

16. The medical pressure applicator device of claim 13, wherein said pressure application area of said disposable tip body is configured to be greater than a cross-sectional area of said work portion of said shaft.

17. The medical pressure applicator device of claim 13, wherein said pressure application area of said disposable tip body is configured to be less than a cross-sectional area of said work portion of said shaft.

18. The medical pressure applicator device of claim 13, wherein said pressure application area of said disposable tip body is configured to form a convex shaped pressure surface.

19. The medical pressure applicator device of claim 13, wherein said pressure application area of said exterior portion of said disposable tip body is configured to form a concave shaped pressure surface.

20. The medical pressure applicator device of claim 13, wherein said pressure application area of said disposable tip body has a plurality of bump protrusions.
